(19) United States
(12) Patent Application Publication
(10) Pub. No.: US 2009/0156870 A1
(43) Pub. Date: Jun. 18, 2009

(54) PROCESS FOR THE CONVERSION OF ETHANE TO MIXED LOWER ALKANES TO AROMATIC HYDROCARBONS

(76) Inventors: Ann Marie Lauritzen, Houston, TX (US); Ajay Madhav Madgavkar, Katy, TX (US)

Correspondence Address: SHELL OIL COMPANY, P O BOX 2463, HOUSTON, TX, 772522463, US

(21) Appl. No.: 12/332,147

(22) Filed: Dec. 10, 2008

Related U.S. Application Data
(60) Provisional application No. 61/012,953, filed on Dec. 12, 2007.
(60) Provisional application No. 61/085,261, filed on Jul. 31, 2008.

(51) Int. Cl. C07C 15/04; C07C 37/08; C07C 15/067; F25J 3/00

(52) U.S. Cl. 585/24; 585/413; 568/798; 62/619

(57) ABSTRACT

A process for producing aromatic hydrocarbons which comprises a) contacting ethane or mixed lower alkanes with an aromatic hydrocarbon conversion catalyst to produce reaction products including benzene, b) separating methane, hydrogen, and C₂₋₅ hydrocarbons from the reaction products of step a), and c) hydrodealkylating the remaining reaction products to produce benzene. In a preferred embodiment, the feed is split into two streams, one of which is catalytically or thermally cracked to produce ethylene which is then combined with the remaining ethane or lower alkanes and contacted with the aromatic hydrocarbon conversion catalyst.

### CROSSREFERENCE TO RELATED APPLICATIONS

This application claims priority to U.S. Provisional Application Ser. No. 61/012,953 filed Dec. 12, 2007 and U.S. Provisional Application Ser. No. 61/085,261 filed Jul. 31, 2008, the entire disclosures of which are hereby incorporated by reference.

### FIELD OF THE INVENTION

The present invention relates to a process for producing aromatic hydrocarbons from ethane or mixed lower alkanes. More specifically, the invention relates to a process for increasing the production of benzene from ethane or a mixture of lower alkanes in a dehydroaromatization process.

### BACKGROUND OF THE INVENTION

There is a projected global shortage for benzene which is needed in the manufacture of key petrochemicals such as styrene, phenol, nylon and polyurethanes, among others. Generally, benzene and other aromatic hydrocarbons are obtained by separating a feedstock fraction which is rich in aromatic compounds, such as reformates produced through a catalytic reforming process and pyrolysis gasolines produced through a naphtha cracking process, from non-aromatic hydrocarbons using a solvent extraction process.

To meet this projected supply shortage, numerous catalysts and processes for on-purpose production of aromatics (including benzene) from alkanes containing six or less carbon atoms per molecule have been investigated. These catalysts are usually bifunctional, containing a zeolite or molecular sieve material to provide acidity and one or more metals such as Pt, Ga, Zn, Mo, etc. to provide dehydrogenation activity. For example, U.S. Pat. No. 4,350,835 describes a process for converting ethane-containing gaseous feeds to aromatics using a crystalline zeolite catalyst of the ZSM-5-type family containing a minor amount of Ga. As another example, U.S. Pat. No. 7,186,871 describes aromatization of C₁-C₄ alkanes using a catalyst containing Pt and ZSM-5.

The ease of conversion of individual alkanes to aromatics increases with increasing carbon number. Hence, aromatization schemes involving methane and/or ethane as principle feed components may include more-reactive hydrocarbons such as olefins, and/or paraffins of higher carbon number than the principle feed component(s), to lower the temperature required to achieve a desired conversion level. For example, U.S. Pat. No. 5,936,135 describes a process for making aromatics from a feed containing one or more lower alkanes by combining this feed with at least one olefin and/or at least one higher paraffin and passing this mixture over a bifunctional pentasil zeolite catalyst at a pressure in the range of 100 to 500 kPa and a temperature in the range of 300 to 600° C. An essential feature of this process is the treatment of the catalyst with a mixture of hydrogen, steam, and an optional inert gas at a temperature ranging from 400 to 800° C., followed by a treatment in air or oxygen at 400 to 800° C., prior to exposure of the catalyst to the feed stream.

Some commercial dehydrocyclodimerization processes for making aromatics from alkanes, such as the process described in U.S. Pat. No. 5,258,563, make a benzene product that contains excessive amounts of non-aromatic C₆₊ hydrocarbons which make it unsuitable for use in some petrochemical processes such as styrene or cyclohexane production. This patent describes additional process steps wherein the product flows into a fractionation zone which is operated at conditions such that the majority of the C₆₊ non-aromatic hydrocarbons along with a portion of the benzene product is removed via an overhead stream. The overhead process stream from this fractionation zone flows to a conversion zone, along with a hydrogen-rich gas, where the non-aromatic C₆₊ hydrocarbons are converted to light (C₁₋₂) hydrocarbons.

It would be advantageous to provide a light alkane dehydroaromatization process wherein (a) a hydrocarbon feed additive or additive mixture that facilitates conversion is optionally generated as part of the process, (b) the catalyst employed does not require a complicated pretreatment, (c) the ultimate yield of benzene is greater than that of any other single aromatic product, and (d) little or no C₅₊ non-aromatic hydrocarbons is produced.

### SUMMARY OF THE INVENTION

The present invention provides a process for producing aromatic hydrocarbons, specifically benzene, which comprises:

(a) optionally splitting an ethane feed into first and second ethane streams,

(b) optionally catalytically or thermally cracking the first ethane stream or the mixed lower alkane feed to produce ethylene and/or a mixture of lower alkylenes,

(c) optionally combining the ethylene or lower alkylenes with the second ethane stream or the mixed lower alkane feed, and

(d) contacting ethane or the combined stream or the mixed lower alkane feed with an aromatic hydrocarbon conversion catalyst to product reaction products including benzene.

In a preferred embodiment, methane, hydrogen and C₂₋₅ hydrocarbons are removed from the reaction products of step

(d). In another preferred embodiment, the remaining products are hydrodealkylated to produce benzene. In another preferred embodiment, the reaction products remaining after the methane, etc. separation are subjected to separation to remove benzene and the remaining reaction products are hydrodealkylated to produce additional benzene. In other embodiments, the benzene is reacted with propylene to make phenol or with ethylene to make ethylbenzene and then styrene.

### BRIEF DESCRIPTION OF THE DRAWINGS

FIG. 1 is a flow diagram which illustrates the production of ethylene and combination of ethylene and ethane in the reactor to produce benzene.

FIG. 2 is a flow diagram which illustrates the ethane or mixed lower alkanes to benzene reaction followed by hydrodealkylation of the non-benzene aromatic products.

FIG. 3 is a flow diagram which illustrates the ethane or mixed lower alkanes to benzene reaction followed by hydrodealkylation of the aromatic products.

FIG. 4 is a flow diagram which illustrates a stacked reactor scheme wherein all of the products of the ethane or mixed lower alkanes to benzene reaction are hydrodealkylated.

FIG. 5 is a flow diagram which illustrates the production of propylene and benzene and their combination to produce phenol.

FIG. 6 is a flow diagram which illustrates the production of propylene and benzene and their combination to make phenol wherein the benzene is separated from the toluene and xylene which are hydrodealkylated.

FIG. 7 is a flow diagram which illustrates the production of propylene and benzene, toluene, and xylene, the hydrodealkylation of the aromatic products to produce benzene and the combination of benzene and propylene to produce phenol.

FIG. 8 is a flow diagram which illustrates the production of propylene and benzene wherein the entire reaction products of the ethane or mixed lower alkanes to benzene reactor are hydrodealkylated to produce benzene and separate C₉₊ products.

FIG. 9 is a flow diagram which illustrates the production of ethylene and benzene and their combination to produce ethylbenzene and then styrene.

FIG. 10 is a flow diagram which illustrate the production of ethylene and benzene followed by hydrodealkylation of the non-benzene aromatic products to produce more benzene which is then combined with ethylene to produce ethylbenzene and then styrene.

FIG. 11 is a flow diagram which illustrates the production of ethylene and the production of benzene, toluene, and xylene which are then hydrodealkylated to produce benzene which is combined with the ethylene to make ethylbenzene and then styrene.

FIG. 12 illustrates the production of ethylene and ethane to benzene reaction products which are hydrodealkylated to produce benzene which is combined with the ethylene to make ethylbenzene and then styrene.

### DETAILED DESCRIPTION OF THE INVENTION

The present invention is a process for producing aromatic hydrocarbons which comprises bringing a hydrocarbon feedstock containing at least about 50 percent by weight of ethane or other C₂ hydrocarbons or mixed lower alkanes with a catalyst composition suitable for promoting the reaction of such hydrocarbons to aromatic hydrocarbons such as benzene into contact at a temperature of about 550 to about 730° C. and a pressure of about 0.01 to about 0.5 Mpa absolute. The primary desired products of the process of this invention are benzene, toluene and xylene.

The hydrocarbons in the feedstock may be ethane, ethylene, mixed lower alkanes or mixtures thereof. In one preferred embodiment, the majority of the feedstock is ethane and more preferably, from about 0 to about 20 weight percent of the feedstock is comprised of ethylene, preferably about 5 to about 10 weight percent. Too much ethylene may cause an acceptable amount of coking and historically, ethylene has been higher in value than benzene. In this embodiment, the hydrocarbon feedstock preferably contains at least about 30 percent by weight of C₂ hydrocarbons, preferably at least about 60 percent by weight. In another preferred embodiment, the majority of the feedstock may include mixed lower alkanes—ethane, propane, butane, and/or C₅₊ alkanes or any combination thereof. In this embodiment, the majority of the feedstock is ethane and propane. The hydrocarbon feedstock preferably contains at least about 30 percent by weight of ethane, at least about 40 percent by weight of C₂₋₄ hydrocarbons, most preferably at least about 50 percent by weight of C₂₋₄ hydrocarbons. In either embodiment, the feedstock may contain in addition other open chain hydrocarbons containing between 3 and 8 carbon atoms as coreactants. Specific examples of such additional coreactants are propane, propylene, n-butane, isobutane, n-butenes and isobutene.

A mixed lower alkane stream may contain C₂, C₃, C₄ and/or C₅₊ alkanes and may be, for example, an ethane/propane/butane-rich stream derived from natural gas, refinery or petrochemical streams including waste streams. Examples of potentially suitable feed streams include (but are not limited to) residual ethane and propane from natural gas (methane) purification, pure ethane, propane and butane streams (also known as Natural Gas Liquids) co-produced at a liquefied natural gas (LNG) site, C₂-C₅ streams from associated gases co-produced with crude oil production (which are usually too small to justify building a LNG plant but may be sufficient for a chemical plant), unreacted ethane “waste” streams from steam crackers, and the C₁-C₃ byproduct stream from naphtha reformers (the latter two are of low value in some markets such as the Middle East). The use of mixed lower alkane feed may save considerable energy because the step of separating the individual relatively pure alkanes from each other is eliminated and, in the case of associated gases co-produced with crude oil production, the cost of reinjection of such gases is eliminated. The mixed lower alkane feed may be deliberately diluted with relatively inert gases such as nitrogen and/or with various light hydrocarbons and/or with low levels of additives needed to improve catalyst performance.

Usually natural gas, comprising predominantly methane, enters an LNG plant at elevated pressures and is pre-treated to produce a purified feed stock suitable for liquefaction at cryogenic temperatures. Ethane, propane, butane and other gases are separated from the methane. The purified gas (methane) is processed through a plurality of cooling stages using heat exchangers to progressively reduce its temperature until liquefaction is achieved. The separated gases may be used as the ethane or mixed lower alkanes feed stream of the present invention. The byproduct streams produced by the process of the present invention may have to be cooled for storage or recycle and the cooling may be carried out using the heat exchangers used for the cooling of the purified methane gas.

Any one of a variety of catalysts may be used to promote the reaction of ethane or mixed lower alkanes to aromatic hydrocarbons. One such catalyst is described in U.S. Pat. No. 4,899,006 which is herein incorporated by reference in its entirety. The catalyst composition described therein comprises an aluminosilicate having gallium deposited thereon and/or an aluminosilicate in which cations have been exchanged with gallium ions. The molar ratio of silica to alumina is at least 5:1.

Another catalyst which may be used in the process of the present invention is described in EP 0 244 162. This catalyst comprises the catalyst described in the preceding paragraph and a Group VIII metal selected from rhodium and platinum. The aluminosilicates are said to preferably be MFI or MEL type structures and may be ZSM-5, ZSM-8, ZSM-11, ZSM-12 or ZSM-35.

Other catalysts which may be used in the process of the present invention are described in U.S. Pat. No. 7,186,871 and U.S. Pat. No. 7,186,872, both of which are herein incorporated by reference in their entirety. The first of these patents describes a platinum containing ZSM-5 crystalline zeolite synthesized by preparing the zeolite containing the aluminum and silicon in the framework, depositing platinum on the zeolite and calcining the zeolite. The second patent describes such a catalyst which contains gallium in the framework and is essentially aluminum-free.

Additional catalysts which may be used in the process of the present invention include those described in U.S. Pat. No. 5,227,557, hereby incorporated by reference in its entirety. These catalysts contain an MFI zeolite plus at least one noble metal from the platinum family and at least one additional metal chosen from the group consisting of tin, germanium, lead, and indium.

One preferred catalyst for use in this invention is described in U.S. Provisional Application No. 61/029481, filed Feb. 18, 2008 entitled “Process for the Conversion of Ethane to Aromatic Hydrocarbons.” This application is hereby incorporated by reference in its entirety. This application describes a catalyst comprising: (1) about 0.005 to about 0.1 % wt (% by weight) platinum, based on the metal, preferably about 0.01 to about 0.05 % wt, (2) an amount of an attenuating metal selected from the group consisting of tin, lead, and germanium, which is no more than 0.02 % wt less than the amount of platinum, preferably not more than about 0.2 % wt of the catalyst, based on the metal; (3) about 10 to about 99.9 % wt of an aluminosilicate, preferably a zeolite, based on the aluminosilicate, preferably about 30 to about 99.9 % wt, preferably selected from the group consisting of ZSM-5, ZSM-11, ZSM-12, ZSM-23, or ZSM-35, preferably converted to the H+ form, preferably having a SiO₂/Al₂O₃ molar ratio of from about 20:1 to about 80:1, and (4) a binder, preferably selected from silica, alumina and mixtures thereof.

Another preferred catalyst for use in this invention is described in U.S. Provisional Application No. 61/029939, filed Feb. 20, 2008 entitled “Process for the Conversion of Ethane to Aromatic Hydrocarbons.” This application is hereby incorporated by reference in its entirety. The application describes a catalyst comprising: (1) about 0.005 to about 0.1 % wt (% by weight) platinum, based on the metal, preferably about 0.01 to about 0.06 % wt, most preferably about 0.01 to about 0.05 % wt, (2) an amount of iron which is equal to or greater than the amount of the platinum but not more than about 0.50 % wt of the catalyst, preferably not more than about 0.20 % wt of the catalyst, most preferably not more than about 0.10 % wt of the catalyst, based on the metal; (3) about 10 to about 99.9 % wt of an aluminosilicate, preferably a zeolite, based on the aluminosilicate, preferably about 30 to about 99.9 % wt, preferably selected from the group consisting of ZSM-5, ZSM-11, ZSM-12, ZSM-23, or ZSM-35, preferably converted to the H+ form, preferably having a SiO₂/Al₂O₃ molar ratio of from about 20:1 to about 80:1, and (4) a binder, preferably selected from silica, alumina and mixtures thereof.

Another preferred catalyst for use in this invention is described in U.S. Provisional Application No. 61/029478, filed Feb. 18, 2008 entitled “Process for the Conversion of Ethane to Aromatic Hydrocarbons.” This application is hereby incorporated by reference in its entirety. This application describes a catalyst comprising: (1) about 0.005 to about 0.1 wt % (% by weight) platinum, based on the metal, preferably about 0.01 to about 0.05 % wt, most preferably about 0.02 to about 0.05 % wt, (2) an amount of gallium which is equal to or greater than the amount of the platinum, preferably no more than about 1 wt %, most preferably no more than about 0.5 wt %, based on the metal; (3) about 10 to about 99.9 wt % of an aluminosilicate, preferably a zeolite, based on the aluminosilicate, preferably about 30 to about 99.9 wt %, preferably selected from the group consisting of ZSM-5, ZSM-11, ZSM-12, ZSM-23, or ZSM-35, preferably converted to the H+ form, preferably having a SiO₂/Al₂O₃ molar ratio of from about 20:1 to about 80:1, and (4) a binder, preferably selected from silica, alumina and mixtures thereof.

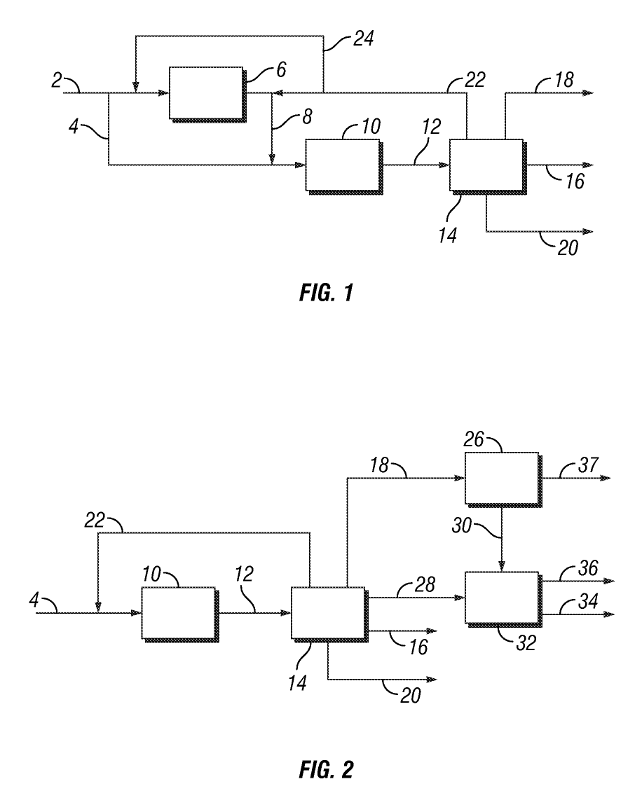

One preferred embodiment of the present invention is shown in FIG. 1. An ethane feed 2 is divided into two streams, the first feed stream is introduced into a catalytic ethane cracker 6 which produces ethylene stream 8. Ethylene stream 8 is combined with the second ethane feed stream 4 and introduced into the ethane to benzene reactor 10 (hereinafter in the description of the figures, the description will refer to ethane but this is intended to include mixed lower alkanes). Introduction of ethylene into the ethane to benzene reactor 10 increases the total hydrocarbon conversion rate and/or the aromatics yield. The reaction products are transferred through line 12 into a separator. The separator removes methane and hydrogen through line 18 and unreacted ethane and ethylene through line 22 which optionally may be recycled to line 8. A portion of line 22 may optionally be diverted in line 24 and combined with the first feed stream which enters the catalytic cracker 6. Optionally, C₉₊ materials may also be separated in separator 14 and leave through line 20. Finally, benzene is produced and leaves separator 14 through line 16.

Another preferred embodiment of the present invention is described in FIG. 2. An ethane feed 4 (which may or may not be partially diverted through an ethane cracker) is introduced into the ethane to benzene reactor 10. The reaction products are fed through line 12 into the separator 14. Optionally, ethane and ethylene are recycled through line 22 to feed line 4. Optionally, C₉₊ materials may be separated through line 20. Benzene is produced and leaves the separator 14 through line 16. Toluene and xylene are also separated from the benzene and leave through line 28 and are introduced into a hydrodealkylation reactor 32 wherein the toluene and xylene are reacted with hydrogen to produce more benzene which leaves through line 34. Light ends materials leave the hydrodealkylation reactor 32 through line 36. The methane and hydrogen stream 18 from separator 14 is optionally treated in hydrogen recovery unit 26 wherein the hydrogen is recovered and is optionally introduced into the hydrodealkylation reactor 32 through line 30. Methane is recovered from the hydrogen recovery unit through line 37.

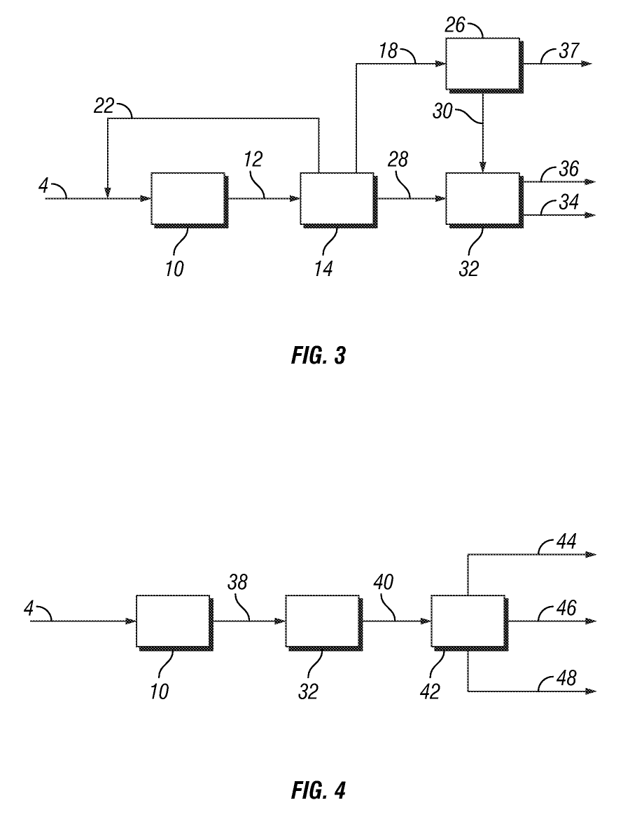

FIG. 3 describes an alternative embodiment to that described in FIG. 2. In this embodiment, the xylene and toluene are not separated from benzene. Instead all three leave the ethane to benzene reactor 14 through line 28 and are introduced into the hydrodealkylation reactor 32. This embodiment provides the advantage of eliminating a separation step and increasing overall benzene yield.

FIG. 4 describes another preferred embodiment of the present invention which has a stacked reactor configuration and wherein all of the reaction products of the ethane to benzene reaction are introduced into the hydrodealkylation reactor 32. Ethane feed 4 enters ethane to benzene reactor 10 and the reaction products leave through line 38 and are introduced into the hydrodealkylation reactor 32. The hydrodealkylation reaction products are introduced through line 40 into separator unit 42. Primary product benzene leaves through line 46. The gases and light materials leave through line 44 and the heavy materials which comprise C₇₊ aromatics leave through line 48. This embodiment has the advantage eliminating several separation steps and increasing overall benzene yield.

The unreacted methane and byproduct C₂₋₅ hydrocarbons may be used in other steps, stored and/or recycled. It may be necessary to cool these byproducts to liquefy them. When the ethane or mixed lower alkanes originate from an LNG plant as a result of the purification of the natural gas, at least some of these byproducts may be cooled and liquefied using. the heat exchangers used to liquefy the purified natural gas (methane).

The hydrodealkylation reaction involves the reaction of toluene, xylenes, ethylbenzene, and higher aromatics with hydrogen to strip alkyl groups from the aromatic ring to produce additional benzene and light ends including methane and ethane which are separated from the benzene. This step substantially increases the overall yield of benzene and thus is highly advantageous.

Both thermal and catalytic hydrodealkylation processes are known in the art. Thermal dealkylation may be carried out as described in U.S. Pat. No. 4,806,700, which is herein incorporated by reference in its entirety. Hydrodealkylation operation temperatures in the described thermal process may range from about 500 to about 800° C. at the inlet to the hydrodealkylation reactor. The pressure may range from about 2000 kPa to about 7000 kPa. A liquid hourly space velocity in the range of about 0.5 to about 5.0 based upon available internal volume of the reaction vessel may be utilized. Due to the exothermic nature of the reaction, it is often required to perform the reaction in two or more stages with intermediate cooling or quenching of the reactants. Two or three or more reaction vessels may therefore be used in series. The cooling may be achieved by indirect heat exchange or interstage cooling. When two reaction vessels are employed in the hydrodealkylation zone, it is preferred that the first reaction vessel be essentially devoid of any internal structure and that the second vessel contain sufficient internal structure to promote plug flow of the reactants through a portion of the vessel.

Alternatively, the hydrodealkylation zone may contain a bed of a solid catalyst such as the catalyst described in U.S. Pat. No. 3,751,503, which is herein incorporated by reference in its entirety. Another possible catalytic hydrodealkylation process is described in U.S. Pat. No. 6,635,792, which is herein incorporated by reference in its entirety. This patent describes a hydrodealkylation process carried out over a zeolite-containing catalyst which also contains platinum and tin or lead. The process is preferentially performed at temperatures ranging from about 250° C. to about 600° C., pressures ranging from about 0.5 MPa to about 5.0 MPa, liquid hydrocarbon feed rates from about 0.5 to about 10 hr ⁻¹ weight hourly space velocity, and molar hydrogen/hydrocarbon feedstock ratios ranging from about 0.5 to about 10.

The integrated process of this invention may also include the reaction of benzene with propylene to produce cumene which may in turn be converted into phenol and/or acetone. The propylene may be produced separately in a propane dehydrogenation unit or may come from olefin cracker process vent streams or other sources.

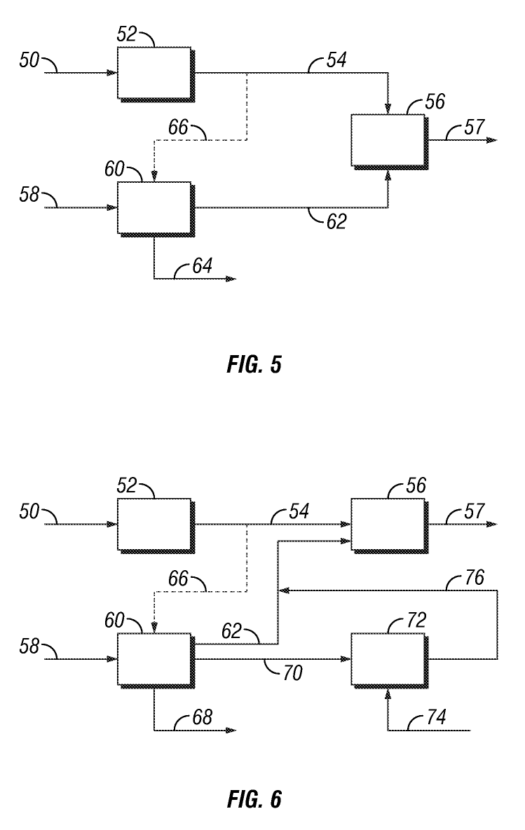

FIG. 5 illustrates one embodiment of the phenol production aspect of the present invention. Propane is introduced through line 50 into propane dehydrogenation unit 52 and propylene made therein flows through line 54 into phenol production system 56. Phenol production system 56 may be comprised of any of the processes for making phenol from benzene such as those described below. Product phenol leaves the phenol production system 56 through line 57.

Ethane flows through line 58 into the ethane to benzene reactor 60 and benzene produced therein flows through line 62 into phenol production system 56. Toluene, xylene and C₉₊ aromatics are separated and leave through line 64. These may be further processed to produce more benzene if desired. Optionally, a portion of the propylene may be introduced into the ethane to benzene reactor through line 66. Line 66 could be a vent stream containing dilute amounts of propylene which is typically low valued. Addition of propylene to the ethane to benzene reactor could increase total hydrocarbon rate and/or aromatics yield.

In the embodiment described in FIG. 6, the reaction is carried out in the same manner except that the toluene and xylene are separated from the benzene and hydrodealkylated to produce additional benzene. The products of the ethane to benzene reactor are separated into benzene which flows through line 62 into phenol production system 56, toluene and xylene which flow through line 70 into hydrodealkylation unit 74, and a C₉₊ aromatics stream which leaves reactor 60 through line 68. The xylene and toluene are hydrodealkylated to produce more benzene which leaves the hydrodealkylation unit 72 through line 76 and is combined with the benzene from ethane to benzene reactor 60. Hydrogen or methane plus hydrogen are introduced into hydrodealkylation reactor 72 through line 74. Product phenol leaves the phenol production system 56 through line 57.

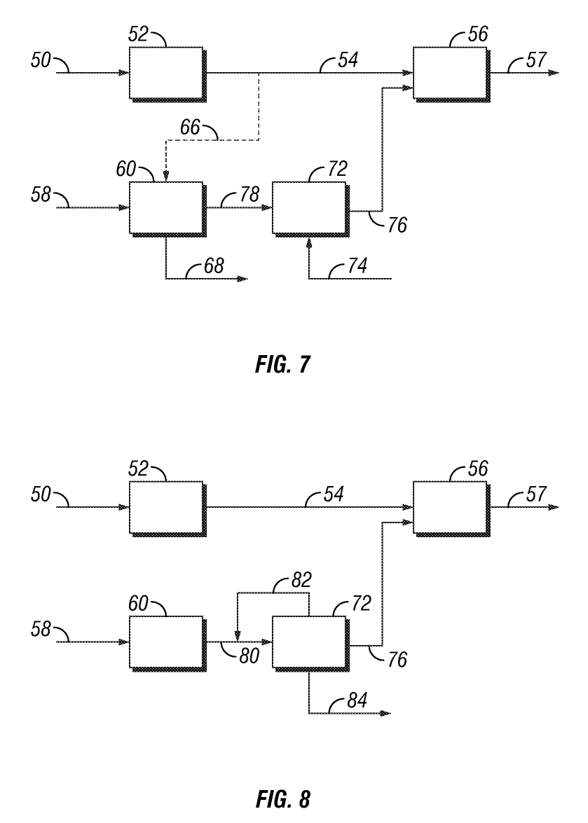

In another embodiment as shown in FIG. 7, no attempt is made to separate the benzene, toluene and xylene components and their mixture is sent directly to the hydrodealkylation unit 72 through line 78 from ethane to benzene reactor 60. Product benzene then flows through line 76 into the phenol production system 56.

In another embodiment as shown in FIG. 8, no attempt is made at separating any of the ethane to benzene reaction products which are sent as a single stream through line 80 to the hydrodealkylation unit 72. Hydrogen or methane plus hydrogen produced in hydrodealkylation reactor 72 is recycled through line 82 into ethane to benzene products stream 80. C₉₊ hydrocarbons which are separated from the benzene leave the hydrodealkylation reactor 72 through line 84.

Phenol can be made from the partial oxidation of benzene or benzoic acid, by the cumene process or by the Raschig process. It can also be found as a product of coal oxidation.

The cumene process is an industrial process for developing phenol and acetone from benzene and propylene in which cumene is the intermediate material during the process. This process converts two relatively inexpensive starting materials, benzene and propylene, into two more valuable ones, phenol and acetone. Other reactants required are oxygen from air and small amounts of a radical initiator. Most of the worldwide production of phenol and acetone is now based on this method.

Cumene is the common name for isopropylbenzene. Nearly all the cumene that is produced as a pure compound on an industrial scale is converted to cumene hydroperoxide which is an intermediate in the synthesis of other industrially important chemicals such as phenol and acetone.

Cumene was for many years been produced commercially by the alkylation of benzene with propylene over a Friedel-Crafts catalyst, particularly solid phosphoric acid or aluminum chloride such as described in U.S. Pat No. 4,343,957. More recently, however, zeolite-based catalyst systems have been found to be more active and selective for propylation of benzene to cumene. It is known that aromatic hydrocarbons can be alkylated in the presence of acid-treated zeolite. U.S. Pat. No. 4,393,262 (1983) teaches that cumene is prepared by the alkylation of benzene with propylene in the presence of a specified zeolite catalyst. U.S. Pat. No. 4,992,606 describes the use of MCM-22 zeolite in the alkylation of benzene with propylene. Other methods are described in U.S. Pat. Nos. 4,441,990, 5,055,627 , 6,525,236 and 6,888,037. All of these patents are herein incorporated by reference in their entirety.

In one embodiment, cumene may be produced by contacting benzene with propylene in a distillation column reactor containing a fixed bed acidic catalytic distillation structure comprising a molecular sieve in a distillation reaction zone thereby catalytically reacting the benzene and propylene to produce an alkylated benzene product including cumene. Cumene may be produced in the catalyst bed under 0.25 to 50 atmospheres of pressure and at temperatures in the range of 50° C. to 500° C., using as the catalyst a mole sieve characterized as acidic. Propylene may be fed to the catalyst bed while benzene may be conveniently added through a reflux to result in a molar excess present in the reactor to that required to react with propylene, thereby reacting substantially all of the propylene and recovering benzene as the principal overhead and cumene and diisopropyl benzene in the bottoms. Concurrently, in the fixed bed the resultant alkylated benzene product is fractionated from the unreacted materials and cumene is separated from the alkylated benzene product (preferably by fractional distillation).

The principal alkylated benzene product is cumene. In addition there may be other alkylated products including di and tri isopropyl benzene, n-propyl benzene, ethyl benzene, toluene, diethyl benzene and di-n-propyl benzene, which are believed to be disproportion and isomerization products of cumene. In a preferred process the residual alkylated products remaining after cumene separation may be passed to a transalkylation reactor operated under conditions to transalkylate polyalkylated benzene, e.g., diisopropyl benzene and triisopropyl benzene, to cumene which may be separated from the other materials in the transalkylation product stream and may be combined with the cumene from the first separation.

Cumene may be oxidized in slightly basic conditions in presence of a radical initiator which removes the tertiary benzylic hydrogen from cumene and hence forms a cumene radical. This cumene radical then bonds with an oxygen molecule to give the cumene hydroperoxide radical, which in turn forms cumene hydroperoxide (C₆H₅C(CH₃) ₂-O—O—H) by abstracting benzylic hydrogen from another cumene molecule. This cumene hydroperoxide converts into cumene radicals and feeds back into subsequent chain formations of cumene hydroperoxides. A pressure of at least about 5 atm may be used to ensure that the unstable peroxide is kept in liquid state.

For example, cumene hydroperoxide may be made according to the process described in U.S. Pat. No. 7,141,703, which is herein incorporated by reference in its entirety. The process comprises providing an oxidation feed consisting essentially of an organic phase. The oxidation feed comprises one or more alkylbenzenes such as cumene and a quantity of neutralizing base having a pH of from about 8 to about 12.5 in 1 to 10 wt. % aqueous solution. The quantity of neutralizing base is effective to neutralize at least a portion of acids formed during the oxidation. The oxidation feed comprises up to an amount of water effective to increase neutralization of acids formed during the oxidation without forming a separate aqueous phase. The oxidation feed is exposed to oxidation conditions effective to produce an oxidation product stream comprising one or more product hydroperoxides.

Cumene hydroperoxide may then be hydrolyzed in an acidic medium to give phenol and acetone.

Additional technologies such as benzene sulfonation/hydrolysis and benzene chlorination/hydrolysis processes may also be used to convert the benzene into phenol, although currently they are not as economically competitive as the cumene process.

The direct oxidation of benzene using air or oxygen is another way in which benzene may be converted into phenol according to the present invention. It does not require reaction with propylene. For example, U.S. Pat. No. 4,992,600, which is herein incorporated by reference in its entirety, describes a process for the oxidation of benzene to phenol which comprises contacting and thereby reacting benzene and oxygen with a (poly)metal salt of a dihydrodihydroxyanthracene(poly)sulfonate having at least one sulfonate moiety on the 2, 3, 6 or 7 position(s) and which salt is dissolved in water, optionally in the presence of an oxidation catalyst, and subsequently separating from the reaction product phenol and the corresponding (poly)metal salt of anthraquinone-(poly)sulfonate. The by-product anthraquinone salt is suitably recycled to the benzene oxidation step by hydrogenating the anthraquinone salt, preferably dissolved in water, to the dihydrodihydroxyanthracene salt by contacting it with hydrogen in the presence of a hydrogenation catalyst.

Additionally, U.S. Pat. No. 6,900,358, which is herein incorporated by reference in its entirety, describes a process for the oxidation of benzene to phenol which comprises continuously contacting, in a distillation column reactor comprising a reaction zone and a distillation zone, benzene with a zeolite catalyst and an oxidant at a temperature in the range of from above 100° C. to 270° C. thereby producing a hydroxylated product, wherein at least a portion of the benzene being in a liquid phase; continuously separating the hydroxylated product from the un-reacted benzene in the distillation zone under conditions effective to vaporize said un-reacted benzene and maintain the hydroxylated product in a liquid phase; and recovering the hydroxylated product from the distillation column reactor.

The integrated process of this invention may also include the reaction of benzene with olefins such as ethylene. The ethylene may be produced separately in an ethane dehydrogenation unit or may come from olefin cracker process vent streams or other sources.

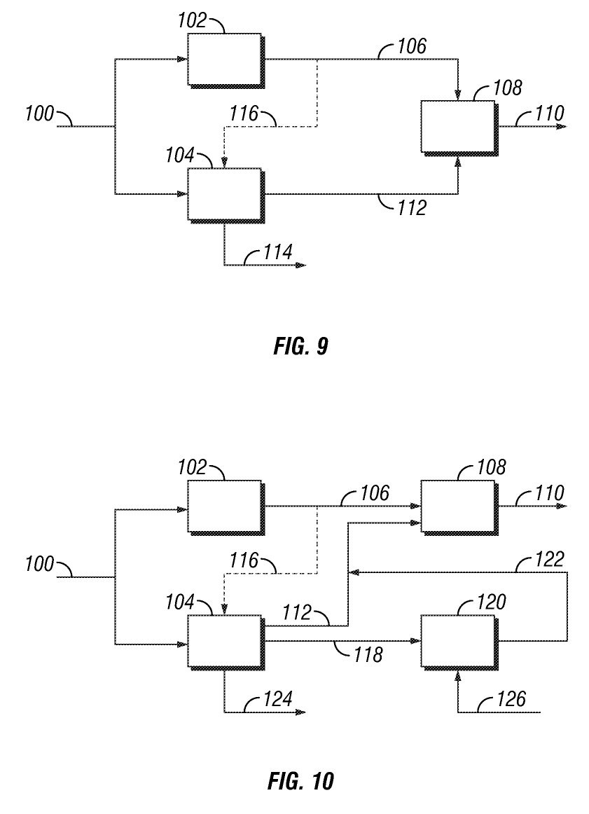

In the embodiment illustrated in FIG. 9, ethane feed line 100 is split into two streams. One stream flows into ethane cracker 102 and the other into ethane to benzene reactor 104. Ethylene leaves cracker 102 through line 106 and flows into styrene production system 108 from which styrene is produced and leaves through line 110. Styrene production system 108 may involve any of the processes described below for reacting ethylene and benzene to product ethylbenzene and then styrene.

Benzene produced in ethane to benzene reactor 104 flows through line 112 into styrene production system 108. Toluene, xylene and C₉₊ aromatics are separated out and leave reactor 104 through line 114. These materials may be further processed to produce more benzene. Some ethylene may be taken from line 106 and introduced into reactor 104 through line 116. As described above, this can be used to increase the total hydrocarbon conversion rate and/or aromatics yield in the reactor 104.

In the embodiment illustrated in FIG. 10, benzene is separated from xylene and toluene in ethane to benzene reactor 104 and the toluene and xylene flow through line 118 into hydrodealkylation unit 120 to produce benzene which flows through line 122 and is combined with benzene in line 112 for introduction into the styrene production system 108. C₉₊ aromatics are separated from the other aromatic materials in ethane to benzene reactor 104 and leave through line 124. Hydrogen or methane plus hydrogen are introduced into the hydrodealkylation unit 120 through line 126.

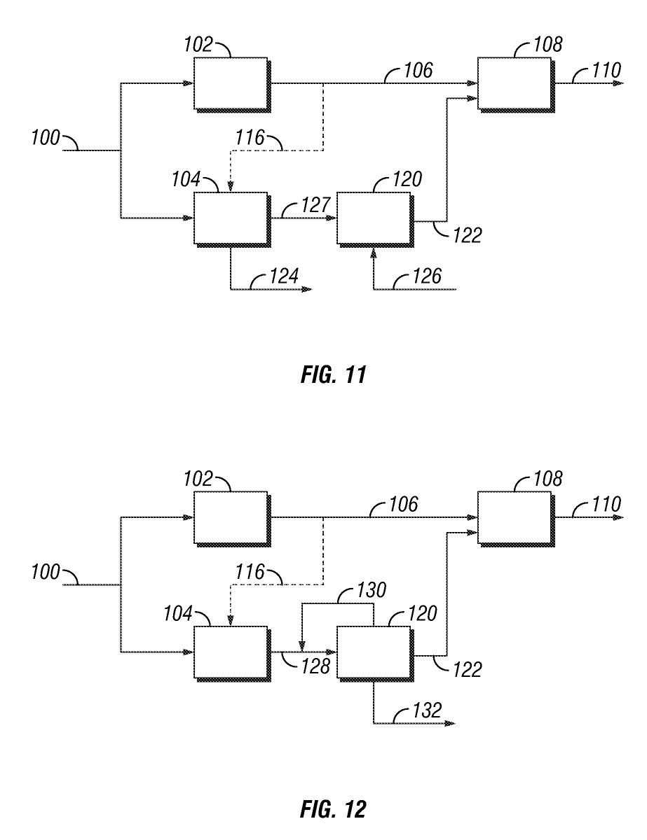

In another embodiment as shown in FIG. 11, no attempt is made to separate the benzene, toluene and xylene components and their mixture is sent to the hydrodealkylation unit 120 in line 127. Benzene made in unit 120 flows through line 122 to styrene product system 108.

In another embodiment as shown in FIG. 12, no attempt is made at separating any of the primary reactor products which are sent as a single stream through line 128 to hydrodealkylation unit 120. Benzene produced therein flows through line 122 to styrene product system 108. Hydrogen or methane plus hydrogen produced in the hydrodealkylation reaction is recycled through line 130 to line 128. C₉₊ hydrocarbons are separated from benzene and are removed through line 132.

Ethylbenzene is an organic chemical compound which is an aromatic hydrocarbon. Its major use is in the petrochemical industry as an intermediate compound for the production of styrene, which in turn is used for making polystyrene, a commonly used plastic material. Although often present in small amounts in crude oil, ethylbenzene is produced in bulk quantities by combining the petrochemicals benzene and ethylene in an acidically-catalyzed chemical reaction. Catalytic dehydrogenation of the ethylbenzene then gives hydrogen gas and styrene, which is vinylbenzene. Ethylbenzene is also an ingredient in some paints.

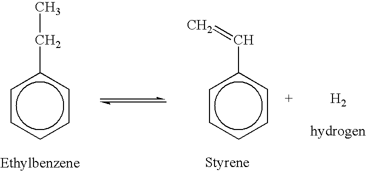

Ethylbenzene may, for example, be produced according to the process of U.S. Pat. No. 5,243,116, which is herein incorporated by reference in its entirety. The process comprises alkylating benzene by contacting the benzene with ethylene in the presence of a catalyst consisting essentially of an acidic mordenite zeolite having a silica/alumina molar ratio of at least 30:1 and a crystalline structure which is determined by X-ray diffraction to be a matrix of Cmcm symmetry having dispersed therein domains of Cmmm symmetry and having a Symmetry Index of at least about 1.

Another process for producing ethylbenzene from benzene is described in U.S. Pat. No. 5,877,370, which is herein incorporated by reference in its entirety. The process comprises passing benzene, ethylene, and a diluent comprising at least one phenyl group and at least one ethyl group to an alkylation zone; reacting the benzene and the ethylene in the alkylation zone in the presence of zeolite beta to alkylate the benzene to form ethylbenzene; and withdrawing from the alkylation zone a product comprising ethylbenzene.

Styrene may then be produced by dehydrogenating the ethylbenzene. One process for producing styrene is described in U.S. Pat. No. 4,857,498, which is herein incorporated by reference in its entirety. Another process for producing styrene is described in U.S. Pat. No. 7,276,636, which is herein incorporated by reference in its entirety. This process for producing styrene comprises: a) reacting benzene and a polyethylbenzene in a transalkylation reactor to form ethylbenzene; b) dehydrogenating ethylbenzene in a dehydrogenation reactor to form styrene; c) withdrawing a dehydrogenation reactor effluent comprising styrene from the dehydrogenation reactor, and passing at least a portion of the dehydrogenation reactor effluent to a dehydrogenation separation section; d) recovering styrene from the dehydrogenation separation section; e) introducing a first inhibitor element component to the dehydrogenation separation section; f) recovering from the dehydrogenation separation section a recycle stream comprising a second inhibitor element component; and g) passing at least 33% of the second inhibitor element component recovered in f) to the transalkylation reactor.

### EXAMPLES

### Example 1

The conceptual example provided below is intended to illustrate but not limit the scope of the invention.

In this example, ethane is converted into aromatic hydrocarbons in an amount of 3000 tons per day using a process configuration as shown in FIG. 1. Thus, 3000 tons/day ethane stream containing minor amounts of propane, butane and methane shown in stream 2 is divided into two streams. The first stream of about 600 tons/day is introduced into a thermal ethane cracker 6 operated at typical process conditions of about 850° C., 0.3 MPa and steam to hydrocarbon ratio of about 0.3. The resulting product mixture in stream 8, containing about 50% ethylene, 30% ethane and balance being mainly methane and hydrogen is combined with the remaining ethane feed in stream 4 and sent to the ethane to benzene reactor 10. Additionally, a recycle stream 22 in an amount of about 1300 tons/day containing primarily ethane and other components such as ethylene, propane, propylene, methane and hydrogen is also combined with stream 8 which is being processed in the ethane aromatization reactor zone. In this non-limiting, illustrative example, the reactor is operated at about 675° C. and 0.15 MPa with a feed rate of about 2 Hr⁻¹ WHSV (Weight Hourly Space Velocity, or 2 tons of feed per ton of catalyst per hour).

The ethane aromatization reactor may feature a fixed bed of catalyst particles catalyst bed over which the ethane-containing feed flows, or a moving- or fluidized-bed reactor in which the catalyst particles cycle slowly or rapidly, respectively, between a reaction zone where aromatization of the feed takes place and a regeneration zone where accumulated coke deposits formed on the catalyst surface under aromatization reaction conditions are removed by controlled combustion in an oxygen-containing atmosphere. Examples of such coke burn/regeneration procedures are described in U.S. Pat. Nos. 4,724,271, 4,705,908, and 5,053,570, which are herein incorporated by reference in their entirety.

The catalyst in the reactor zone preferentially comprises a crystalline acidic zeolitic material, one or more metal dehydrogenation components in minor amount(s), and an optional binder material such as silica or alumina. For example, the catalyst may comprise 0.5 to 5.0% w gallium plus about 65% w ZSM-5 zeolite having a silica:alumina molar ratio of 40, with an alumina binder. Catalysts of this type are described in U.S. Pat. No. 4,350,835 referred to above. Prior to initial exposure to the reaction feed, and/or after a coke burn off step, the catalyst may optionally be treated at elevated temperature with air, nitrogen, hydrogen, steam, or dilute hydrogen sulfide in hydrogen or nitrogen, or by any chemically compatible mixtures thereof, or by a sequential procedure involving exposure to first one, then another of these. Examples of possible catalyst treatment schemes are described in U.S. Pat. Nos. 4,613,716, 4,120,910, 4,808,763, 5,157,183, and 7,186,782, which are all herein incorporated be reference in their entirety.

The reaction products in amount approximately 4300 tons/day are transferred through line 12 to a product separation section 14 which consists of compressors, vapor-liquid separators, distillation columns etc. In this section, majority of methane and hydrogen is removed from the products as stream 18 in an approximate amount 900 tons/day and is processed further downstream if required. The unreacted ethane and ethylene along with any remaining C₁₋₅ hydrocarbons are sent for recycle in stream 22 mentioned earlier. Benzene is recovered and leaves the separation section 14 though stream 16 in an amount approximately 1000 tons/day. Other byproducts such as toluene, xylenes, and C₉₊ aromatics in an approximate amount 1100 tons/day leave the separation section 14 though stream 20.

### Example 2

Catalysts A and B were made with low levels of Pt and Ga on extrudate samples containing 80% wt of CBV 2314 ZSM-5 powder (23:1 molar SiO2:Al2O3 ratio, available from Zeolyst International) and 20% wt alumina binder. These catalysts were prepared as described in U.S. Provisional Application No. 61/029478, filed Feb. 18, 2008 entitled “Process for the Conversion of Ethane to Aromatic Hydrocarbons.” The extrudate samples were calcined in air up to 650° C. to remove residual moisture prior to use in catalyst preparation. The target metal loadings for catalyst A were 0.025% w Pt and 0.09% wt Ga. The target metal loadings for catalyst B were 0.025% wt Pt and 0.15% wt Ga.

Metals were deposited on 25-50 gram samples of the above ZSM-5/alumina extrudate by first combining appropriate amounts of stock aqueous solutions of tetraammine platinum nitrate and gallium(III) nitrate, diluting this mixture with deionized water to a volume just sufficient to fill the pores of the extrudate, and impregnating the extrudate with this solution at room temperature and atmospheric pressure. Impregnated samples were aged at room temperature for 2-3 hours and then dried overnight at 100° C.

Catalysts made on the ZSM-5/alumina extrudate were tested “as is,” without crushing. For each performance test, a 15-cc charge of fresh (not previously tested) catalyst was loaded into a Type 316H stainless steel tube (1.40 cm i.d.) and positioned in a four-zone furnace connected to a gas flow system.

Prior to performance testing, the catalyst charges were pretreated in situ at atmospheric pressure (ca. 0.1 MPa absolute) as follows:
(a) calcination with air at 60 liters per hour (L/hr), during which the reactor wall temperature was increased from 25 to 510° C. in 12 hrs, held at 510° C. for 4-8 hrs, then further increased from 510 to 630° C. in 1 hr, then held at 630° C. for 30 min;
(b) nitrogen purge at 60 L/hr, 630° C. for 20 min;
(c) reduction with hydrogen at 60 L/hr, for 30 min, during which time the reactor wall temperature was raised from 630° C. to the temperature used for the actual run.

At the end of the above reduction step, the hydrogen flow was terminated, and the catalyst charge was exposed to a feed consisting of 67.2% wt ethane and 32.8% wt propane at atmospheric pressure (ca. 0.1 MPa absolute), 650-700° C. reactor wall temperature, and a feed rate of 500-1000 GHSV (500-1000 cc feed per cc catalyst per hr). Three minutes after introduction of the feed, the total reactor outlet stream was sampled by an online gas chromatograph for analysis. Based on composition data obtained from the gas chromatographic analysis, initial ethane, propane and total conversions were computed according to the following formulas:

ethane conversion, %=100×(% wt ethane in feed−% wt ethane in outlet stream)/(% wt ethane in feed)

propane conversion, %=100×(% wt propane in feed−% wt propane in outlet stream)/(% wt propane in feed)

total ethane+propane conversion=((% wt ethane in feed×% ethane conversion)+(% wt propane in feed×% propane conversion))/100

Table 1 lists the results of online gas chromatographic analyses of samples of the total product streams of these reactors taken at 3 minutes after introduction of the feed. Under these conditions, over 99% wt of the propane in the feed and over 55% w of the ethane in the feed was converted in all of these catalyst performance tests. The product stream contains benzene and higher aromatics, along with hydrogen and light hydrocarbons, including some ethane which can be recycled. Furthermore, it can be seen that only very small amounts of C₄ non-aromatic hydrocarbons were produced and no C₅ non-aromatic hydrocarbons were produced.

TABLE 1

| Catalyst | A | B | B | A | B | A |
|---|---|---|---|---|---|---|
| Catalyst charge weight, g | 11.58 | 11.52 | 12.36 | 11.43 | 11.51 | 11.73 |
| Reactor Wall Temperature, ° C. | 650 | 675 | 675 | 700 | 700 | 700 |
| Total feed rate, GHSV | 500 | 600 | 1000 | 800 | 800 | 1000 |
| Total feed rate, WHSV | 0.89 | 1.07 | 1.67 | 1.44 | 1.43 | 1.76 |
| % Ethane Conversion | 56.38 | 71.07 | 58.22 | 77.16 | 77.05 | 65.77 |
| % Propane Conversion | 99.3 | 99.48 | 99.11 | 99.61 | 99.61 | 99.5 |
| Total % (Ethane + Propane) Conversion | 70.51 | 80.4 | 71.64 | 84.53 | 84.45 | 76.84 |

Reactor Outlet Composition, % wt

| | | | | | | |
|---|---|---|---|---|---|---|
| Hydrogen | 5.31 | 6.29 | 5.71 | 6.48 | 6.54 | 5.99 |
| Methane | 17.91 | 19.28 | 16.36 | 20.47 | 20.25 | 17.13 |
| Ethylene | 2.11 | 3.83 | 2.89 | 5.76 | 5.45 | 6.65 |
| Ethane | 29.26 | 19.43 | 28.06 | 15.34 | 15.42 | 22.99 |
| Propylene | 0.22 | 0.33 | 0.32 | 0.46 | 0.43 | 0.67 |
| Propane | 0.23 | 0.17 | 0.29 | 0.13 | 0.13 | 0.16 |
| C4 | 0.02 | 0.02 | 0.03 | 0.05 | 0.05 | 0.09 |
| C5 | 0 | 0 | 0 | 0 | 0 | 0 |
| Benzene | 26.97 | 29.68 | 27.45 | 30.06 | 30.34 | 24.99 |
| Toluene | 8.15 | 8.28 | 8.21 | 7.97 | 7.92 | 8.09 |
| C8 Aromatics | 0.74 | 0.83 | 0.79 | 0.94 | 0.88 | 1.06 |
| C9+ Aromatics | 9.06 | 11.86 | 9.88 | 12.33 | 12.58 | 12.17 |
| Total Aromatics | 44.93 | 50.84 | 46.33 | 51.31 | 51.73 | 46.31 |

### Example 3

Using fresh (not previously tested) charges of catalysts A and B described in Example 2 additional performance tests were conducted as described in Example 2 except that the feed consisted of 32.8% w ethane and 67.2% w propane. Table 2 lists the results of online gas chromatographic analyses of samples of the total product streams of these reactors taken at 3 minutes after introduction of the feed. Under these conditions, over 99% wt of the propane in the feed and over 20% w of the ethane in the feed was converted in all of these catalyst performance tests. The product stream contains benzene and higher aromatics, along with hydrogen and light hydrocarbons, including some ethane which can be recycled. Furthermore, it can be seen that only very small amounts of C₄ non-aromatic hydrocarbons were produced and no C₅ non-aromatic hydrocarbons were produced.

TABLE 2

| Catalyst | A | B | B | B | B | A |
|---|---|---|---|---|---|---|
| Catalyst charge weight, g | 11.58 | 11.51 | 11.52 | 11.93 | 12.36 | 11.73 |
| Reactor Wall Temperature, ° C. | 650 | 675 | 675 | 675 | 675 | 700 |
| Total feed rate, GHSV | 500 | 500 | 600 | 800 | 1000 | 800 |
| Total feed rate, WHSV | 0.99 | 0.98 | 1.22 | 1.57 | 1.9 | 1.6 |
| % Ethane Conversion | 23.73 | 59.12 | 48.81 | 42.53 | 36.17 | 66.32 |
| % Propane Conversion | 99.65 | 99.84 | 99.78 | 99.74 | 99.68 | 99.85 |
| Total % (Ethane + Propane) Conversion | 74.55 | 86.38 | 83.09 | 81 | 78.88 | 88.86 |

Reactor Outlet Composition, % wt

| | | | | | | |
|---|---|---|---|---|---|---|
| Hydrogen | 4.79 | 5.7 | 5.45 | 5.61 | 5.78 | 5.78 |
| Methane | 19.65 | 23.7 | 22.34 | 19.64 | 17.05 | 24.54 |
| Ethylene | 2.88 | 2.95 | 3.1 | 3.67 | 4.17 | 4.44 |
| Ethane | 25.21 | 13.51 | 16.77 | 18.83 | 20.91 | 11.03 |
| Propylene | 0.27 | 0.21 | 0.27 | 0.38 | 0.45 | 0.37 |
| Propane | 0.23 | 0.11 | 0.14 | 0.17 | 0.21 | 0.1 |
| C4 | 0.03 | 0.01 | 0.02 | 0.05 | 0.06 | 0.04 |
| C5 | 0 | 0 | 0 | 0 | 0 | 0 |
| Benzene | 27.23 | 31.73 | 30.71 | 29.14 | 26.99 | 29.44 |
| Toluene | 9.28 | 7.75 | 8.77 | 9.82 | 10.17 | 7.69 |
| C8 Aromatics | 1.08 | 0.71 | 0.9 | 1.19 | 1.4 | 0.91 |
| C9+ Aromatics | 9.34 | 13.61 | 11.52 | 11.51 | 12.82 | 15.66 |
| Total Aromatics | 46.93 | 53.8 | 51.9 | 51.65 | 51.37 | 53.69 |

What is claimed is:

1. A process for producing aromatic hydrocarbons which comprises:
a) splitting an ethane feed or a mixed lower alkanes feed into first and second ethane or mixed lower alkanes streams,
b) catalytically or thermally cracking the first ethane or mixed lower alkanes stream to produce ethylene or mixed lower alkenes,
c) combining the ethylene or mixed lower alkenes with the second ethane or mixed lower alkanes stream, and
d) contacting the combined stream with an aromatic hydrocarbon conversion catalyst to produce reaction products including benzene.

2. The process of claim 1 wherein methane, hydrogen and C₂₋₅ hydrocarbons are removed from the reaction products of step d).

3. The process of claim 2 wherein the products of claim 2 are hydrodealkylated to produce benzene.

4. The process of claim 2 wherein benzene is separated from the products of claim 2 and the remaining reaction products are hydrodealkylated to produce additional benzene.

5. The process of claim 2 wherein C₉₊ reaction products are separated from the products of claim 2 and the remaining reaction products are hydrodealkylated to produce benzene.

6. The process of claim 5 wherein benzene is also separated from the products of claim 2.

7. The process of claim 2 wherein C₈₊ reaction products are separated from the products of claim 2 and the remaining reaction products are hydrodealkylated to produce benzene.

8. The process of claim 7 wherein benzene is also separated from the products of claim 2.

9. The process of claim 1 wherein the reaction products of step d) are hydrodealkylated to produce benzene.

10. The process of claim 9 wherein a stacked bed reactor system is used.

11. The process of claim 3 wherein the hydrogen removed from the reaction products of step d) is used to hydrodealkylate the products of claim 3.

12. The process of claim 1 wherein the reaction products of step d) are hydrodealkylated and both steps are carried out in the same reactor.

13. A process for producing aromatic hydrocarbons which comprises:
a) contacting ethane or mixed lower alkanes with an aromatic hydrocarbon conversion catalyst to produce reaction products including benzene,
b) separating methane, hydrogen, and C₂₋₅ hydrocarbons from the reaction products of step a), and
c) hydrodealkylating the remaining reaction products to produce benzene.

14. The process of claim 13 wherein the separation in step b) includes separation of benzene produced in step a) and the remaining reaction products hydrodealkylated in step c) comprise toluene and xylene.

15. The process of claim 14 wherein C₉₊ reaction products are also separated in the separation of step b).

16. A process for producing aromatic hydrocarbons which comprises:
a) contacting ethane or mixed lower alkanes with an aromatic hydrocarbon conversion catalyst to produce reaction products including benzene,
b) hydrodealkylating all of the reaction products from step a), and
c) separating benzene from the reaction products of step b) from gases including methane, hydrogen and lower C₂₋₅ hydrocarbons and heavy aromatic hydrocarbons.

17. A process for producing phenol which comprises producing benzene according to the process of claim 1 and then either:
1) reacting benzene with propylene to produce cumene, oxidizing the cumene to produce cumene hydroperoxide and then hydrolyzing the cumene hydroperoxide in an acidic medium to produce phenol, or
2) directly oxidizing benzene using air or oxygen, or
3) sulfonating the benzene and then hydrolyzing the sulfonate product, or
4) chlorinating the benzene and the hydrolyzing the chlorinated product to produce phenol.

18. A process for producing styrene which comprises producing benzene according to the process of claim 1, reacting the benzene with ethylene to produce ethylbenzene and then dehydrogenating the ethylbenzene to produce styrene.

19. A process for producing aromatic products from natural gas which comprises introducing natural gas to a liquefied natural gas plant at elevated pressures, pre-treating the natural gas by removing therefrom ethane and/or a mixture of lower alkanes to produce a purified feed stock suitable for liquefaction at cryogenic temperatures, processing the purified gas through a plurality of cooling stages using heat exchangers to progressively reduce its temperature until liquefaction is achieved, and producing aromatic products from ethane or mixture of lower alkanes according to the process of claim 1.

20. The process of claim 19 wherein at least some of the unreacted methane and byproduct C₂₋₅ hydrocarbons are cooled using the heat exchangers used to liquefy the purified natural gas.
